(19) United States
(12) Patent Application Publication
(10) Pub. No.: US 2007/0233245 A1
(43) Pub. Date: Oct. 4, 2007

(54) Methods and instruments for delivering intervertebral devices

(75) Inventor: Hai H. Trieu, Cordova, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/394,959

(22) Filed: Mar. 31, 2006

(51) Int. Cl. A61F 2/44

(52) U.S. Cl. 623/17.11

(57) ABSTRACT

A method comprises selecting an intervertebral implant having a predetermined shape with a height dimension and a width dimension. The implant further comprises an empty or partially filled reservoir. The method further comprises selecting a deformation instrument and inserting the intervertebral implant into the deformation instrument. The deformation instrument comprises at least one joint for moving the deformation instrument between an expanded state and a collapsed state. The method further comprises placing the intervertebral implant in the deformation instrument while in the expanded state and activating the at least one joint to move the deformation instrument into a collapsed state. The method further comprises collapsing the reservoir to reduce one of the dimensions of the implant and moving the implant from the deformation instrument into an intervertebral disc space. The implant is allowed to expand substantially to the predetermined shape within the intervertebral disc space. The method further comprises filling the reservoir and maintaining within the reservoir a volume of filling material.

### BACKGROUND

Within the spine, the intervertebral disc functions to stabilize and distribute forces between vertebral bodies. The nucleus pulposus is surrounded and confined by the annulus fibrosis.

Intervertebral discs are prone to injury and degeneration. For example, herniated discs are common, and typically occur when normal wear, or exceptional strain, causes a disc to rupture. Degenerative disc disease typically results from the normal aging process, in which the tissue gradually looses its natural water and elasticity, causing the degenerated disc to shrink and possibly rupture.

Intervertebral disc injuries and degeneration are frequently treated by replacing or augmenting the existing disc material. Current methods and instrumentation used for treating the disc require a relatively large hole to be cut in the disc annulus to allow introduction of the implant. After the implantation, the annulus must be plugged, sewn closed, or other wise blocked to avoid allowing the implant to be expelled from the disc. Besides weakening the annular tissue, creation of the opening and the subsequent repair adds surgical time and cost. A need exists for instrumentation and methods for implanting an intervertebral implant using minimally invasive surgical techniques.

### SUMMARY

In one embodiment, a method comprises selecting an intervertebral implant having a predetermined shape with a height dimension and a width dimension. The implant further comprises an empty or partially filled reservoir. The method further comprises selecting a deformation instrument and inserting the intervertebral implant into the deformation instrument. The deformation instrument comprises at least one joint for moving the deformation instrument between an expanded state and a collapsed state. The method further comprises placing the intervertebral implant in the deformation instrument while in the expanded state and activating the at least one joint to move the deformation instrument into a collapsed state. The method further comprises collapsing the reservoir to reduce one of the dimensions of the implant and moving the implant from the deformation instrument into an intervertebral disc space. The implant is allowed to expand substantially to the predetermined shape within the intervertebral disc space. The method further comprises filling the reservoir and maintaining within the reservoir a volume of filling material.

In another embodiment, a method for treating an intervertebral disc space comprises selecting an elastic body, the body having a first end, a second end, a central portion, and a first configuration wherein said first end and said second end are positioned adjacent to said central portion to form at least one inner fold, wherein the elastic body has a surface that includes wrinkles, indents or projections that relieve stress and prevent cracking or tearing of the implant when the implant is straightened for implantation and further wherein the elastic body comprises a reservoir for receiving a filling material. The method further comprises inserting the elastic body into a deformation instrument and collapsing the reservoir of the elastic body with the deformation instrument to configure the elastic body into a second, straightened configuration. The method further comprises inserting the elastic body in the second, straightened configuration through an opening in an intervertebral disc annulus fibrosis into the intervertebral disc space, wherein the body returns to said first configuration after said insertion. The method further comprising filling the reservoir with reservoir with a filling material capable of setting.

In another embodiment, a system for implanting an intervertebral implant into an intervertebral disc space comprises a deformation instrument adapted to receive the implant and adapted to deform an inner reservoir of the implant from an uncollapsed state to a collapsed state. The system further comprises an insertion instrument adapted move the implant from the deformation instrument to the intervertebral disc space and an injection instrument adapted to receive a volume of filling material and adapted to dispense the volume of filling material into the inner reservoir.

In another embodiment, an intervertebral implant for replacing at least part of a natural nucleus pulposus comprises an outer elastic body having a body volume, a predetermined height and a predetermined width. The implant further comprises at least one inner reservoir located at least partially within the outer elastic body and a volume of settable filling material for filling the inner reservoir The body volume is greater than volume of filling material.

Additional embodiments are included in the attached drawings and the description provided below.

### BRIEF DESCRIPTION OF THE DRAWINGS

FIG. 1 is a sagittal view of a section of a vertebral column.

FIGS. 2-4 are a sequence of views of the implantation of an intervertebral implant including insertion, accessing with a filling instrument, and expanding the implant.

FIGS. 5a-5b are side views of an implantation instrument according to one embodiment of the present disclosure.

FIGS. 6a-6b are side views of an implantation instrument according to another embodiment of the present disclosure.

FIGS. 7a-7b are side views of an implantation instrument according to another embodiment of the present disclosure.

FIGS. 8a-8e, 9a-9b, and 10a-10b are top cross-sectional views of implants according to various embodiments of the present disclosure.

### DETAILED DESCRIPTION

The present disclosure relates generally to methods and instruments for delivering a spinal implant, and more particularly, to methods and instruments for minimally invasive intervertebral device implantation. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

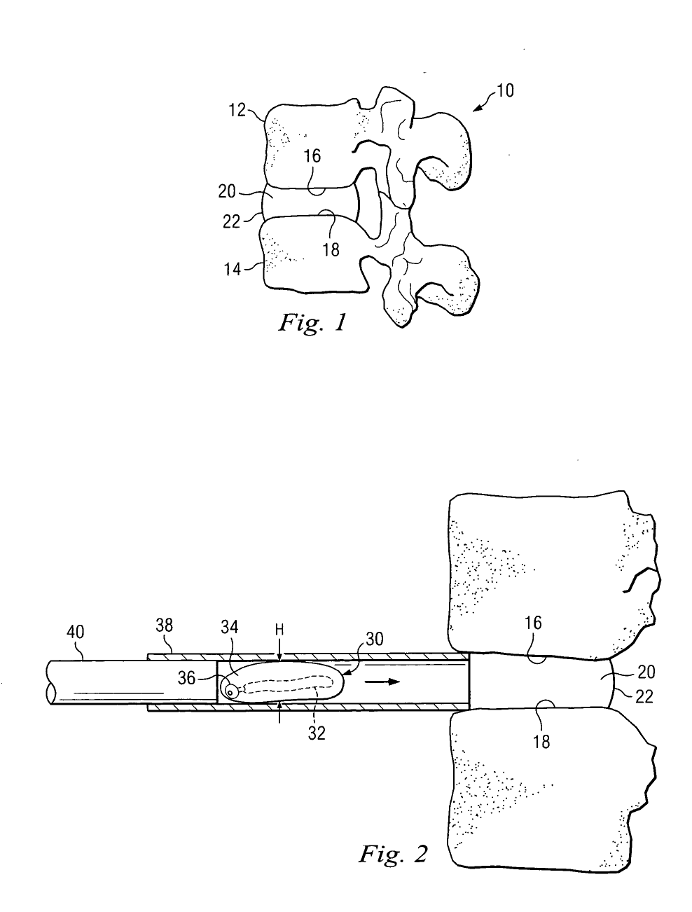

Referring first to FIG. 1, the reference numeral 10 refers to a vertebral joint section or a motion segment of a vertebral column. The joint section 10 includes adjacent vertebral bodies 12, 14. The vertebral bodies 12, 14 include endplates, 16, 18, respectively. An intervertebral disc space 20 is located between the endplates 16, 18, and an annulus 22 surrounds the space 20. In a healthy joint, the space 20 contains a nucleus pulposus.

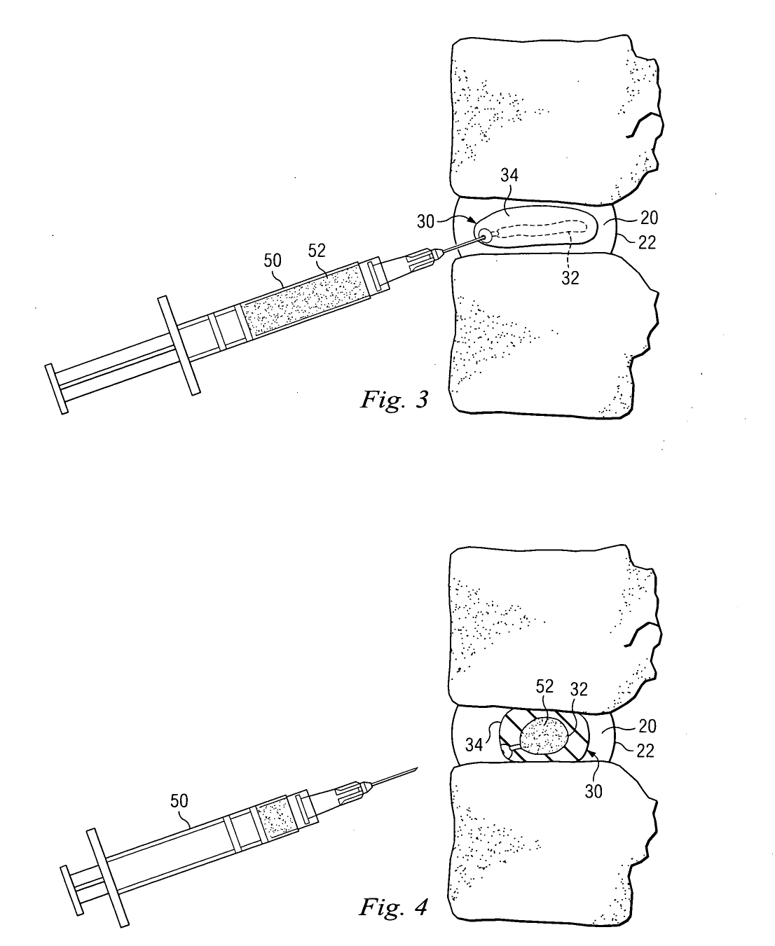

Referring now to FIGS. 2-4, an intervertebral implant 30 may be used to replace all or a portion of the nucleus pulposus and fill all or a portion of the disc space 20. The implant 30 comprises an implant reservoir 32 and an outer portion 34. The implant 30 may further include a valve 36. Throughout this description, the term “reservoir” denotes an empty or at least partially empty void which may be filled with a solid or fluid material. In the embodiment of FIGS. 2-4, the reservoir 32 is formed entirely within at least one external surface of the intervertebral implant 30. In alternative embodiments, reservoirs may also exist when a portion of the reservoir is within at least one external surface of implant (e.g., a depression on an external surface could be a reservoir in the context of the present invention).

To place the intervertebral implant 30, a small incision is first cut in the annulus 22 of the disc being repaired or augmented. The annulus may be accessed through a posterior, lateral, anterior, or any other suitable approach. A guide wire or other small instrument may be used to make the initial hole. If necessary, successively larger holes are cut from an initially small puncture. The hole (also called an aperture, an opening, or a portal, for example) may be as small as possible to minimize expulsion of the material through the hole after the surgery is complete.

Also if necessary, a dilator may be used to dilate the hole, making it large enough to deliver the implant 30 to replace or augment the disc nucleus. The dilator may stretch the hole temporarily and avoid tearing so that the hole can return back to its undilated size after the instrument is removed. Although some tearing or permanent stretching may occur, the dilation may be accomplished in a manner that allows the hole to return to a size smaller than its dilated size after the surgery is complete.

Any of a variety of tools may be used to prepare the disc space, including specialized pituitary rongeurs and curettes for reaching the margins of the nucleus pulposus. Ring curettes may be used to scrape abrasions from the vertebral endplates as necessary. Using these instruments, a centralized, symmetrical space large enough to accept the implant footprint may be prepared in the disc space.

Once a hole is provided, an implant instrument 38, such as a cannula, is inserted into the hole. The implant 30 may be deformed to have a minimal profile by collapsing the reservoir 32. The implant 30 may be deformed by becoming inserted into the implant instrument 38 or may be deformed outside and then inserted into the implant instrument. The partially or completely empty reservoir 32 allows the collapsed height H of the implant 30 to be minimized for passage through the minimally invasive implant instrument 38. An insertion instrument 40, such as a probe, may then be used to push the implant 30 through the cannula and finally into the disc nucleus space 20.

Once inside the prepared disc space, the implant 30 may be allowed to expand such that the height of the uncollapsed implant 30 is greater than the collapsed height H. In an alternative embodiment, the implant may remain collapsed until the reservoir is filled as described below.

After the implant 30 is delivered into the space 20, the implant instrument 38 may be withdrawn. An injection instrument 50, such as a syringe, may be inserted through the annulus 22 and into the valve 36. The valve 36 may be any port in the implant 30 that provides access to the reservoir 32. The valve 36 may be self-sealing, sealable, or pluggable.

The injection instrument 50 may be filled with a volume of biocompatible filling material 52 for filling all or a portion of the reservoir 32. The filling material 52 may be injected from the injection instrument 50, through the valve 36 and into the reservoir 32. As the material flows into the reservoir 32, the implant 30 may expand, distracting the vertebral endplates 16, 18, restoring the disc space 20 to a desired height, and placing the annulus 22 into tension. The filling material 52 may set by curing or polymerizing in situ. The in situ curable materials may cure to a compliant or rigid mass depending upon the materials selected. In other alternatives, the material may remain fluid or gel-like inside the reservoir 32. Biological or pharmaceutical agents may be added to the filling material.

After the material 52 is injected, the injection instrument 50 may be removed, and the hole in the annulus 22 may be allowed to close. Depending upon the size of the remaining aperture in the annulus, a suture, staple, blocking implant, or other type of fastening device may be used to prevent the contents of the space 20 from migrating through the annulus 22.

Examples of filling materials that cure or polymerize in situ include elastomer, hydrogel, or rigid polymer materials. Suitable elastomers may include silicone elastomers, polyurethane elastomers, silicone-polyurethane copolymers, polyolefin rubbers, butyl rubbers, or combinations thereof. Suitable hydrogels may include polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), sulfonated polymers, or combinations thereof. Suitable rigid polymers may include polymethylmethacrylate, silicones, polyurethanes, polyvinyl alcohol, polyamide, aromatic polyamide, polyether, polyesterliquid crystal polymer, ionomer, poly(ethylene-co-methacrylic) acid, PBT (polybutylene terephthalate), polycarbonate, or combinations thereof.

The outer portion 34 of the implant 30 can be comprised of a single material or it can be fabricated from multiple materials. The material or combination of materials chosen may have load bearing properties to provide mechanical support to the spine as well as contain the filling material 52. In addition, the material of the outer portion 34 may have a degree of flexibility to permit relative movement of the vertebral bodies 12, 14 between which the implant 30 is positioned. The outer portion 34 may be formed of an elastic material that may stretch when the reservoir is filled or may be formed of a relatively inelastic material that unfolds to a predetermined shape and does not further stretch when filled with the filling material. One possible material that can provide the mechanical support and containment properties is a thermoplastic silicone polyurethane copolymer material.

The outer portion 34 may, alternatively, be formed from a wide variety of biocompatible polymeric materials, including elastic materials, such as elastomeric materials, hydrogels or other hydrophilic polymers, or composites thereof. Suitable elastomers include silicone, polyurethane, copolymers of silicone and polyurethane, polyolefins, such as polyisobutylene rubber and polyisoprene rubber, neoprene rubber, nitrile rubber, vulcanized rubber and combinations thereof. The vulcanized rubber described herein may be produced, for example, by a vulcanization process utilizing a copolymer produced as described, for example, in U.S. Pat. No. 5,245,098 from 1-hexene and 5-methyl-1,4-hexadiene. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include naturally occurring materials such as glucomannan gel, hyaluronic acid, polysaccharides, such as cross-linked carboxyl-containing polysaccharides, and combinations thereof.

The nature of the materials employed to form the implant 30 may be selected so the formed implants have sufficient load bearing capacity. For example, a compressive strength of about 0.1 Mpa may be suitable, with compressive strengths in the range of about 1 Mpa to about 20 Mpa being particularly suitable.

Either the outer portion of the implant or the filling materials can also be bioresorbable. The outer portion may be a bioresorbable non-porous (sheet or film) or a bioresorbable porous (braided fibers) shell. The filling material may be a precursor of resorbable polymer that polymerizes, cures or crosslinks in situ. The following families of resorbable polymers can be used for the outer portion and/or the filling materials: poly(L-lactic acid), poly(D,L-lactic acid), poly(D L-lactic-co-glycolic acid), poly(glycolic acid), poly(epsilon-caprolactone), polyorthoesters, polyanhydrides, polyhydroxy acids, polydioxanones, polycarbonates, polyaminocarbonates, polyurethane, poly(ethylene glycol), poly(ethylene oxide), partially or fully hydrolyzed poly(vinyl alcohol), poly(ethylene oxide)-co-poly(propylene oxide) block copolymers (poloxamers and meroxapols), poloxamines or combinations thereof.

To provide the desired support to the spinal joint 10, the volume of the outer portion 34 of the implant 30 may be greater than the volume of the filling material 52. Furthermore, as with a natural nucleus, the mechanical support provided by (or load carried by) the outer portion may be greater than that provided by the injectable material 52. Thus the outer portion 34 may be firmer than the filling material 52 inside the reservoir 32.

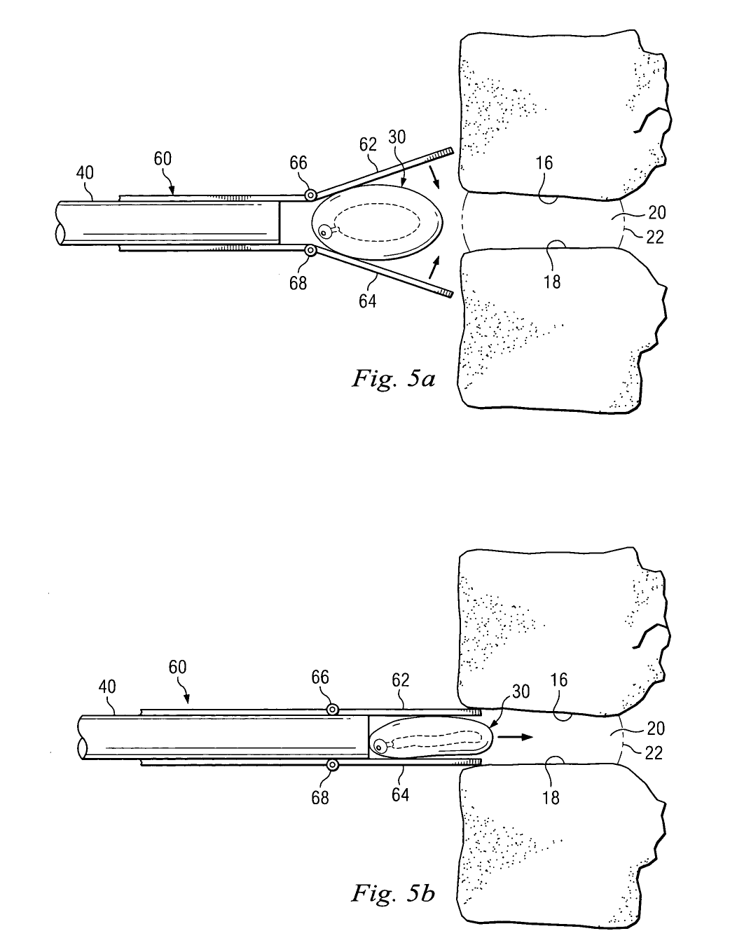

Referring now to FIG. 5a-5b, in an alternative embodiment, an instrument 60 may be used as both a deformation instrument and implant instrument. In this embodiment, the implant 30 is positioned between clamp portions 62, 64 which are pivotable about joint mechanisms 66, 68, respectively, as shown in FIG. 5a. The clamping portions 62, 64 are moved together to collapse the implant 30. A locking mechanism associated with joint mechanism 66, 68 may lock the clamping portions 62, 64 to hold the implant 30 in a collapsed state. With the implantation instrument 60 positioned through the annulus 22 as described above, the insertion instrument 40 may be used to push the implant 30 from between the clamp portions 62, 64 and into the intervertebral disc space 20. Once implanted, the implant 30 may be filled and expanded as described above. This disclosure contemplates a variety of joint mechanisms which may include pivot joints, hinge joints, or any structure that deforms or bends in response to a force.

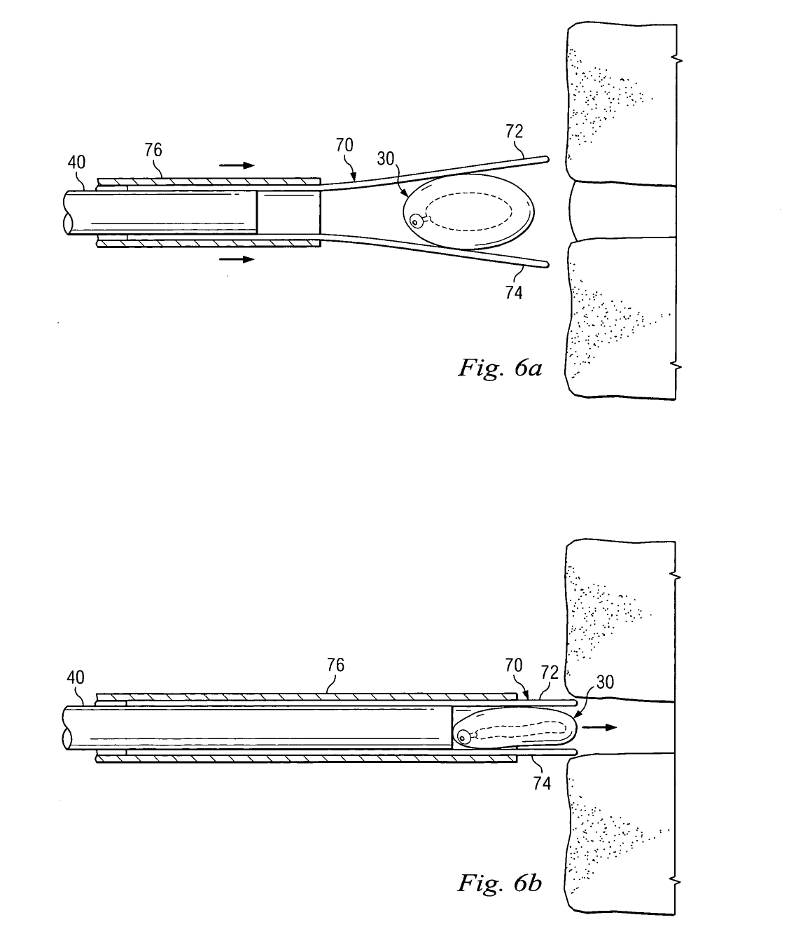

Referring now to FIGS. 6a-6b, a deformation and implantation instrument 70 may comprise a pair of clamping portions 72, 74 the distal end of which are positioned within a guide sleeve 76. In use, the implant 30 may be positioned between the clamping portions 72, 74, and the guide sleeve 76 may be advanced toward the implant 30. As the sleeve 76 is advanced, the clamping portions 72, 74 may move together to collapse the implant 30. With the implantation instrument 70 positioned through the annulus 22 as described above, the insertion instrument 40 may be pushed through the sleeve 76 and the clamping portions 72, 74 to push the implant 30 into the intervertebral disc space 20. Once implanted, the implant 30 may be filled and expanded as described above.

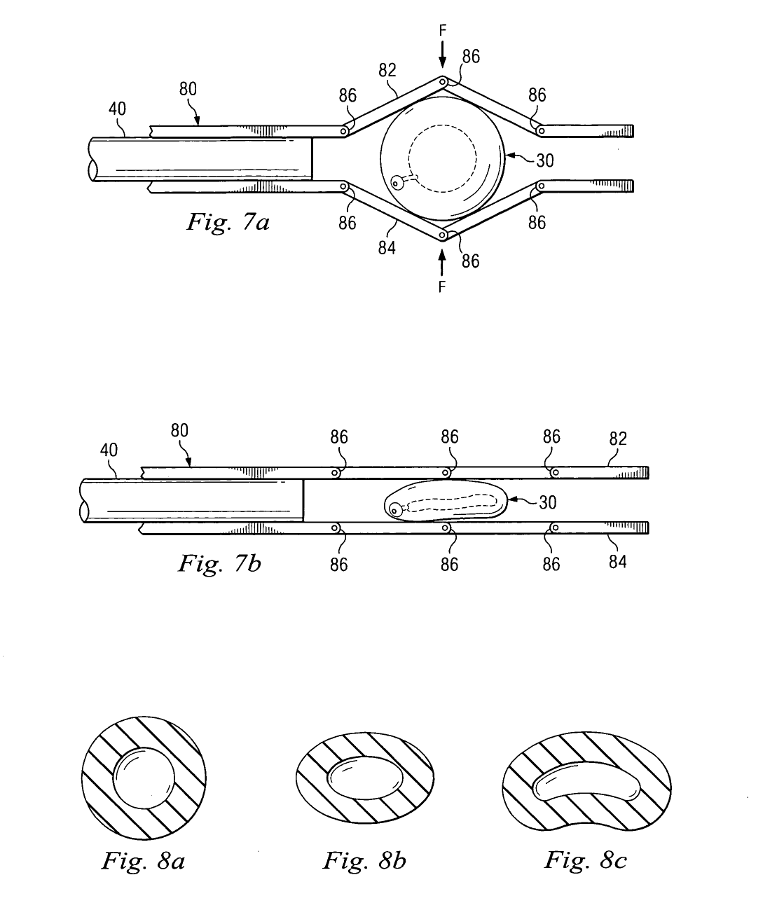

Referring now to FIGS. 7a-7b, a deformation and implantation instrument 80 may comprise a pair of clamping portions 82, 84 having multiple joint mechanisms 86. In use, the implant 30 may be positioned between the clamping portions 82, 84 and a force F may be applied to deform the clamping portions 82, 84 and to collapse the implant 30. With the implant 30 in a collapsed state, the joint mechanisms 86 may be locked into place. With the implantation instrument 80 positioned through the annulus 22 as described above, the insertion instrument 40 may be pushed through the clamping portions 82, 84 to push the implant 30 into the intervertebral disc space 20. Once implanted, the implant 30 may be filled and expanded as described above.

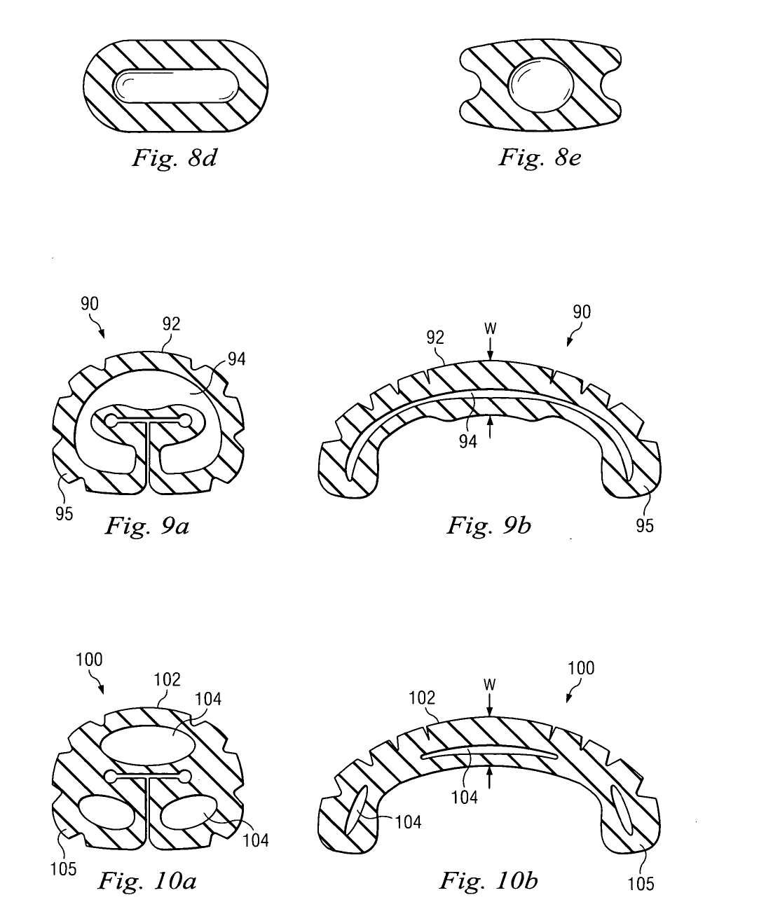

Referring now to FIGS. 8a-8e, various shapes of collapsible implants may be used with any of the implantation or deformation instruments described above. For example, FIG. 8a is a cross-sectional top view of a spherical intervertebral implant having an internal reservoir. FIG. 8b is a cross-sectional top view of an oblong or “football” shaped intervertebral implant having an internal reservoir. FIG. 8c is a cross-sectional top view of a kidney-shaped intervertebral implant having an internal reservoir. FIG. 8d is a cross-sectional top view of a capsular intervertebral implant having an internal reservoir. FIG. 8e is a cross-sectional top view of an irregularly shaped implant having an internal reservoir. The shape of the implant may be selected to achieve a desired amount of distraction, to correct for irregular load placement, or to compensate for incomplete disc space preparation.

Referring now to FIGS. 9a, 9b, 10a, and 10b, a NAUTILUS spinal implant, under development by Medtronic, Inc. of Minneapolis, Minn., may be adapted for low profile insertion by incorporating a reservoir. Spinal implants 90, 100 may have load bearing elastic bodies sized for placement into an intervertebral disc space. The implants 90, 100 may be formed of elastomeric material that allows the implants to return to a folded shape after being unfurled into a generally straightened configuration. The elastic body may have a surface 92, 102 that includes wrinkles, indents or projections that relieve stress and prevent cracking or tearing of the implant when the implant is straightened for implantation. The implants 90, 100 may further include reservoirs 94, 104 surrounded by outer portions 95, 105. Further characteristics which may be found in the implants 90, 100 are described in greater detail in U.S. Pat. No. 6,620,196 or 6,893,466 which are incorporated by reference herein.

Using an implantation and/or deformation instrument as described above or as described in U.S. patent application Ser. No. 10/717,687, which is incorporated by reference herein, the implant 90, 100 may be unfurled into a relatively straight configuration as shown in FIGS. 9b and 10b. In the straightened or unfurled state, the reservoirs may become collapsed, allowing the implant to have a smaller width W than a similar implant without reservoirs would have. As the implant 90, 100 is pushed from the implantation or delivery instrument and into the disc space 20, it is allowed to fold back into its original, unstraightened configuration. Once in place within the annular wall, the implant 90, 100 may be filled completely or partially with a filling material as described above.

The outer portions 95, 105 may be formed of any of the materials described above for outer portion 34 and the filling material for filling the reservoirs 94, 104 may be any of the materials described above for filling material 52. Likewise, the outer portions 95, 105 may have a greater volume than the volume of the reservoirs 94, 104.

The fillable reservoirs 32, 94, 104 allow the respective implants 30, 90, 100 to be customizable to a particular anatomy, eliminating the need to maintain large inventories of various sizes and simplifying the preparation and measurement that must be performed prior to implantation. Furthermore, the reservoirs provide the physician with the flexibility to choose from a variety of filling materials to achieve a desired stiffness in the filled implant. For example, the reservoirs may be filled with a softer material to reduce the compressive modulus, making the implant more compressible than a solid implant without reservoirs. The reduced width during implantation minimizes or avoids resection or violation of the facet joint when delivered via a posterior approach. The preservation of the facet joint will help maximize the stability of the treated level. Reducing the width of the implant during delivery may also minimize the size of the annular defect for better implant retention.

Although the instruments and implants described are suitable for intervertebral applications, it is understood that the same implants and instruments may be modified for use in other regions including an interspinous region or a bone cavity. Furthermore, the instruments and implants of this disclosure may be incorporated in certain aspects into an intervertebral prosthesis device such as a motion preserving artificial disc.

The delivery of any of the implants described above may facilitated by lubricating any of the instruments or any of the implants described above. Suitable lubricants may include oils, solvents, bodily fluids, fat, saline, or hydrogel coatings. For example, in FIG. 2, the interior surface of the cannula 38 may be lubricated to ease the passage of the implant 30. Alternately, the implant 30 could be lubricated.

In an alternative embodiment, any of the implantation instruments disclosed above may be curved or flexible to improve access to the intervertebral disc space.

In this description, height refers to a dimension measured along the longitudinal axis of the vertebral column and width refers to a dimension measured along an axis (such as an anterior-posterior or lateral axis) of a transverse plane.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A method comprising:
selecting an intervertebral implant having a predetermined shape with a height dimension and a width dimension and comprising an empty or partially filled reservoir;
selecting a deformation instrument;
inserting the intervertebral implant into the deformation instrument, wherein the deformation instrument comprises at least one joint for moving the deformation instrument between an expanded state and a collapsed state;
placing the intervertebral implant in the deformation instrument while in the expanded state;
activating the at least one joint to move the deformation instrument into a collapsed state;
collapsing the reservoir to reduce one of the dimensions of the implant;
moving the implant from the deformation instrument into an intervertebral disc space;
allowing the implant to expand substantially to the predetermined shape within the intervertebral disc space; and
filling the reservoir and maintaining within the reservoir a volume of filling material.

2. The method of claim 1 wherein the deformation instrument comprises at least three joints.

3. The method of claim 1 further comprising lubricating the deformation instrument.

4. The method of claim 1 further comprising locking the joint in the collapsed state.

5. The method of claim 1 wherein the step of activating the joint further comprises sliding a guide tube along the deformation instrument to activate the joint.

6. The method of claim 1 wherein the implant further comprises an outer portion surrounding the reservoir, the outer portion having an outer portion volume and further wherein the outer portion volume is greater than the volume of filling material.

7. The method of claim 1 wherein the implant further comprises an outer portion surrounding the reservoir, the outer portion adapted to support a maximum first load and further wherein the volume of filling material is adapted to support a maximum second load less than the maximum first load.

8. The method of claim 1 wherein the predetermined shape is spherical.

9. The method of claim 1 wherein the predetermined shape is kidney shaped.

10. The method of claim 1 wherein the implant further comprises a center portion and opposite arm portions and wherein the opposite arms are folded toward the center portion when in the predetermined shape.

11. The method of claim 10 wherein the opposite arms extend away from the center portion when the reservoir is collapsed.

12. The method of claim 1 wherein the step of filling the reservoir comprises injecting the volume of settable filling material.

13. The method of claim 1 wherein the filling material is an elastomer.

14. The method of claim 1 wherein the filling material is a hydrogel.

15. The method of claim 1 wherein the filling material is a curable rigid polymer.

16. The method of claim 1 wherein the filling material sets to a non-fluid consistency.

17. A method for treating an intervertebral disc space comprising:
selecting an elastic body, the body having a first end, a second end, a central portion, and a first configuration wherein said first end and said second end are positioned adjacent to said central portion to form at least one inner fold, wherein the elastic body comprises a reservoir for receiving a filling material;
inserting the elastic body into a deformation instrument;
collapsing the reservoir of the elastic body with the deformation instrument to configure the elastic body into a second, straightened configuration;
inserting the elastic body in the second, straightened configuration through an opening in an intervertebral disc annulus fibrosis into the intervertebral disc space, wherein the body returns to said first configuration after said insertion; and
filling the reservoir and maintaining within the reservoir a filling material.

18. The method of claim 17 wherein the step of collapsing the reservoir collapses the height of the elastic body.

19. The method of claim 17 wherein the step of collapsing the reservoir collapses the width of the elastic body.

20. The method of claim 17 wherein the deformation instrument comprises a joint for moving the deformation instrument between an expanded state and a collapsed state and the step of collapsing the reservoir of the elastic body with the deformation instrument includes actuating the joint to move the deformation instrument from the expanded state to the collapsed state.

21. The method of claim 17 wherein the elastic body has a surface that includes wrinkles, indents or projections that relieve stress and prevent cracking or tearing of the implant when the implant is straightened for implantation.

22. The method of claim 17 wherein the filling material sets to a non-fluid consistency after entering the reservoir.

23. A system for implanting an intervertebral implant into an intervertebral disc space, the system comprising:
a deformation instrument adapted to receive the implant and adapted to deform an inner reservoir of the implant from an uncollapsed state to a collapsed state;
an insertion instrument adapted move the implant from the deformation instrument to the intervertebral disc space; and
an injection instrument adapted to receive a volume of filling material and adapted to dispense the volume of filling material into the inner reservoir.

24. The system of claim 23 wherein the deformation instrument comprises a jointed body having a maximized configuration for receiving the implant and a minimized configuration for deforming the reservoir to the collapsed state.

25. The system of claim 24 wherein the jointed body includes at least two joints.

26. The system of claim 23 wherein the deformation instrument comprises a guide tube configured to receive the deformation instrument.

27. The system of claim 23 wherein the deformation instrument comprises a clamping portion movable by a joint mechanism.

28. An intervertebral implant for replacing at least part of a natural nucleus pulposus, the implant comprising:
an outer elastic body having a body volume, a predetermined height and a predetermined width;
at least one inner reservoir located at least partially within the outer elastic body; and
a volume of settable filling material for filling the inner reservoir;
wherein the body volume is greater than volume of filling material.

29. The implant of claim 28 wherein the outer elastic body is firmer than the filling material.

30. The implant of claim 28 wherein the filling material develops a consistency firmer than the outer elastic body.

31. The implant of claim 28 wherein the volume of filling material is curable in situ.

32. The implant of claim 28 further comprising a self-sealing valve.

33. The implant of claim 28 further comprising a central portion and a pair of opposite arms, wherein the implant has a deformed state in which the opposite arms extend away from the central portion and a undeformed state in which the opposite arms fold in toward the central portion.

34. The implant of claim 28 wherein the filling material is an elastomer.

35. The implant of claim 28 wherein the filling material is a hydrogel.

36. The implant of claim 28 wherein the filling material is a rigid polymer after setting.

37. The implant of claim 28 wherein the filling material is injectable through a syringe.

38. The implant of claim 28 wherein the implant has a collapsed state in which the collapsed height is smaller than the predetermined height.

39. The implant of claim 28 wherein the implant has a collapsed state in which the collapsed width is smaller than the predetermined width.
